(19) United States
(12) Patent Application Publication
(10) Pub. No.: US 2016/0289626 A1
(43) Pub. Date: Oct. 6, 2016

(54) MICRO-TITRE PLATE WITH CAPILLARY MEMBRANE SUPPLY UNIT

(71) Applicant: 3M INNOVATIVE PROPERTIES COMPANY, St. Paul, MN (US)

(72) Inventors: Florian BONN, Leverkussen (DE); Carsten MEIXNER, Huckeswagen (DE); Haythem KORBI, Wuppertal (DE)

(73) Assignee: 3M INNOVATIVE PROPERTIES COMPANY, St. Paul, MN (US)

(21) Appl. No.: 15/036,128

(22) PCT Filed: Nov. 12, 2014
(86) PCT No.: PCT/EP2014/074310

§ 371 Date: May 12, 2016

(30) Foreign Application Priority Data
Nov. 13, 2013 (EP) 13192645.3

(51) Int. Cl. C12M 1/00; C12M 1/32

(52) U.S. Cl. CPC C12M 29/04; C12M 23/12

(57) ABSTRACT

An apparatus for growing and/or examining cells, comprising the arrangement of at least one bowl-shaped culture dish having a base and a side wall, and an interior which is bounded by the base and side wall and has an opening at the top end, characterized in that—at least one sheet-like capillary membrane system composed of at least one capillary membrane is disposed in the interior of the culture dish,—wherein the at least one capillary membrane has a semipermeable wall, a lumen which is enclosed by the wall and has at least one open end and has a fluid connection via its open end to a supply line having a wall and a lumen, such that liquids, media, gases and/or other substances can be passed through the supply line and the capillary membrane system,—wherein the capillary membrane system is disposed in the region of the base of the respective culture dish and its areal extent is limited by the side wall of the culture dish and—wherein the at least one supply line leads out of the interior through the opening.

The invention relates to a device for growing and studying cells, comprising an arrangement of a plurality of isolated bowl-shaped culture dishes, each having a base and a side wall and an interior space which is bounded by the base and the side wall and which has an opening towards the top.

For studying and cultivating adherent, suspension and hybridoma cells, cell culture plates are often used, which are available, for example, as so-called microtiter plates having 1, 4, 6, 24 or 96 bowl-shaped cell culture dishes, for example, known by the names of 6-, 24-, 96-well plates. Such cell culture plates make it possible, e.g., to quickly screen active substances in cell cultures, for example to assess the effect of cytostatics on cancer or tumor cells of a cancer patient.

Implementing such cell experiments in the laboratory often requires a large number of concurrent experiments, which are for the most part carried out in such microtiter plates. Cultivating cells, in particular the intermittent feeding and removal of media from the depressions (wells) of the titer plate is extremely resource-intensive. Furthermore, supplying the cells with nutrient medium, for example, is often too irregular. The concentration of the ingredients (nutrients, messenger substances, extracellular substances) is therefore subject to substantial fluctuations. This may impair cell development.

EP-A-1 159 444 discloses individual membrane modules suitable for testing a wide variety of active substances in cells. These membrane modules comprise a housing having an interior space which is bounded by a cover, a base and a side wall. In the interior space, at least one system of first capillary membranes and one system of second capillary membranes are arranged, which are disposed in the interior space to form two-dimensional layers. The capillary membranes pass through the side wall of the interior space with at least one of their ends and are separately combined into bundles according to systems and embedded in a casting compound at their at least one end.

DE-A-10 2006 031 871 describes a Petri dish for growing and studying cells. In the interior space of the Petri dish, a hollow fiber membrane system having a plurality of hollow fiber membranes is arranged through which liquids or gases can be conveyed. At their ends, the hollow fiber membranes are combined into bundles and connected to a supply line or discharge line. Via the hollow fiber membrane system, a plurality of circular culture spaces, e.g. six, which are situated consecutively in the running direction of the hollow fiber membranes, can be arranged in the Petri dish so that the hollow fiber membranes run consecutively through a plurality of culture spaces and handle supply to and disposal from a plurality of culture spaces.

DE-A-102 21 565 describes microtiter plates, for example for cultivating cells, in which flat membranes are arranged in the base of the bowl-shaped depressions or culture dishes, via which a common supply device arranged below the culture dishes can supply, e.g., nutrient culture media into the culture dishes and thus to the cells located in said culture dishes.

It is the problem of the present invention to provide a device having a plurality of bowl-shaped culture dishes for growing and studying cells which can be supplied, independently and in a controlled manner with a nutrient solution, for example. At the same time, simple handling and flexible use of the device should be possible.

The problem is solved by a device for growing and studying cells which comprises an arrangement of at least one bowl-shaped culture dish having a base and a side wall, and an interior space which is bounded by the base and the side wall, which has an opening towards the top, wherein the device is characterized in that
at least one planar capillary membrane system consisting of at least one capillary membrane is arranged in the interior space of the at least one culture dish,
wherein the at least one capillary membrane has a semi-permeable wall, a lumen which is enclosed by a wall and at least one open end,
wherein the at least one capillary membrane with its at least one open end is fluidically connected to at least one supply line having a wall and a lumen, so that fluids, media, gases and/or other substances can be conveyed through the supply line and the at least one capillary membrane system,
and wherein the at least one capillary membrane system is arranged in the region of the base of the at least one culture dish and its two-dimensional extension is bounded by the side wall of the at least one culture dish, and
wherein the at least one supply line leads out of the interior space through the opening.

By means of the present device, it is possible to supply the cell cultures in the isolated culture dishes separately without causing mutual interference of the cell cultures. In doing so, the individual cell cultures can also be supplied, e.g., with different media without the media mixing.

The bowl-shaped culture dishes are preferably contained together in microtiter plates, wherein 2-hole, 4-hole, 6-hole, 12-hole, 24-hole plates, etc. (2-, 4-, 6-, 12-, 24-well plates) are customary. In a preferred embodiment, the device therefore comprises at least two culture dishes which are isolated from one another and have no connection to one another and which are separately provided with at least one capillary membrane system. In a further preferred embodiment, the device comprises at least six culture dishes which are isolated from one another and have no connection to one another and which are separately provided with at least one capillary membrane system. The base of the at least one bowl-shaped culture dish preferably has an area in the range of 2 to 30 cm² and particularly preferably an area in the range of 2 to 15 cm². The interior space of the at least one bowl-shaped culture dish is open at its top and bounded by the base and the side wall. The side wall itself preferably does not have an opening and is closed. Base and interior wall are preferably fluid-impermeable, i.e., impermeable to gases and/or liquids. The interior space can have different cross-sectional geometries. Preferably, the interior space has a circular, oval, rectangular or square cross-section.

The at least one culture dish is preferably provided with a cover which covers the opening of the interior space of the culture dish, wherein the at least one supply line can then pass through the cover. In the case of a plurality of culture dishes, the cover may also be a common covering for all of the culture dishes of the device through which the supply lines associated with the individual culture dishes pass in the region of their openings. It has proven to be favorable if connector sleeves are mounted in the cover above the openings of the at least one culture dish, with which the supply lines are connected and via which they pass through the cover and lead out of the interior space. The connector sleeves are preferably glued into the cover, wherein customary adhesives can be used.

The at least one planar capillary membrane system is understood to be an arrangement consisting of at least one capillary membrane which extends two-dimensionally in the region of the base of the culture dish or in its vicinity. In doing so, the at least one planar capillary membrane system can touch the side wall; however, the at least one planar capillary membrane system, or the capillary membrane forming it, does not pass through the side wall or the base. Inside the interior space, the at least one capillary membrane opens into a supply line, which, according to the invention, leads out of the at least one culture dish via the opening of the interior space.

The dimensions of the at least one planar capillary membrane system result from its external measurements in the two-dimensional extension. At a maximum, the at least one planar capillary membrane system can extend across the entire cross-section of the interior space up to the side wall of the bowl-shaped culture dish. Preferably, the at least one planar capillary membrane system extends across 30 to 90%, and particularly preferably across 50 to 80% of the area of the base of the at least one culture dish or cell culture dish. In a likewise preferred embodiment, the at least one planar capillary membrane system can be removed from or added to the interior space. There is no material connection to the base or the side wall of the interior space.

Here, the planar capillary membrane system may consist of a single capillary membrane which is arranged in a meandering fashion. In this embodiment, at least one of the ends of the meandering capillary membranes is open and connected to the supply line. However, the at least one capillary membrane system may also comprise a plurality of meandering capillary membranes which, together with their ends, open into the supply line.

In particular for use in culture dishes having an internal space with a circular or oval cross-section, an embodiment may be advantageous in which the planar capillary membrane system consists of at least one capillary membrane that is arranged spirally in the region of the base of the at least one culture dish. In this embodiment, one of the ends of the at least one spiral capillary membrane is preferably open and connected to the supply line, whereas the other end is closed. The planar capillary membrane system may likewise be composed of a plurality of capillary membranes arranged in mutually concentric circles, the ends of which, in one embodiment, are open and embedded in each case at opposite sides of the wall of the individual supply line.

In a preferred embodiment, the at least one planar capillary membrane system comprises a plurality of mutually parallel capillary membranes and can preferably be composed of 2 to 100 mutually parallel capillary membranes. The number of mutually parallel capillary membranes may particularly preferably be 5 to 50.

The capillary membranes of the mutually parallel capillary membranes are embedded in the supply line at their open ends so fluid-tightly in the wall at its outer periphery that a fluid connection exists between the lumen of the supply line and the lumen of the capillary membranes, and that liquids, media, gases and/or other substances can be conveyed through the supply line and the capillary membranes. The at least one supply line is preferably open at its one end and connectable to a supply unit or a disposal unit, whereas the other end of the at least one supply line is closed. Embedding can be accomplished, for example, with a curable silicone material, a polyurethane resin or an epoxy resin.

Curable silicone materials are preferably used because of their superior flexibility. In the event that the capillary membranes are embedded in a supply line with only one of their ends, the other, opposite, end of the capillary membranes is closed, for example by fusing or bonding. However, the planar capillary membrane system may also be fitted to the contour, for example round, of the bowl-shaped culture dish, for example by appropriately adapted end-fusing of the non-embedded ends of the capillary membranes in capillary membrane systems having only one supply line; this results in an arched contour at this edge of the planar capillary membrane system. The capillary membranes can also be embedded in a supply line at both of their ends on one side of the arrangement, for which purpose the capillary membranes are designed to be U-shaped at their free ends. In those cases, the capillary membranes are operated in the dead-end mode.

In a further embodiment of the capillary membrane system, the capillary membranes are open at both of their ends and embedded with one end in each case in a respective supply line, wherein the supply lines are then preferably located at opposite sides of the planar capillary membrane system. In this case as well, embedding is carried out in such a way that the capillary membranes are fluid-tightly embedded at the outer periphery and create a fluid connection between the lumen of each of the supply lines and the lumen of the capillary membranes. Such an embodiment having two supply lines makes it possible to perfuse the at least one capillary membrane system in the cross-flow mode.

The diameter of the at least one supply line conforms primarily to the external diameter of the capillary membranes embedded in it. The at least one supply line therefore preferably has an internal diameter in the range of 0.1 to 10 mm. It is likewise preferred if the wall thickness of the flexible silicone tube ranges from 0.1 to 5 mm. In the event that a supply line with a non-circular cross-section is used, the equivalent diameter d=4A/U of the internal cross-section is used as the internal diameter, where A is the area of the internal cross-section and U its circumference. For example, the internal cross-section of the supply line may also be oval or approximately square or rectangular. For the at least one supply line, a silicone tube, e.g., through the walls of which the capillary membrane ends pass and into which they are glued has proven to be suitable. Preferably, the at least one common supply line is a flexible silicone tube. Embedding or bonding in the wall of the supply line can be accomplished with conventional adhesives, such as curable silicone materials, polyurethane resins or epoxy resins.

In an advantageous embodiment, the capillary membrane system may be designed in the form of a capillary membrane mat, in which a plurality of mutually parallel capillary membranes are connected to one another by means of spaced and mutually parallel connection elements and are held at a distance from one another by the connection elements. In doing so, the connection elements can run transverse to the mutually parallel capillary membranes or also at another angle. The connection elements can also be adhesive strips or, for example, strand-like elements made of a silicone material. In a preferred embodiment, the capillary membranes are connected by means of thread-like connection elements to form a mat. Particularly preferably, the connection elements are multifilament textile threads. Multifilament polyester threads, polypropylene threads or polytetrafluoroethylene threads have proven to be particularly successful multifilament textile threads.

In a preferred embodiment, the capillary membrane mat can be a knitted mat in which the capillary membranes and the connecting fibers are knitted together and in which the capillary membranes run transverse to the direction of extension of the capillary membrane mat and in which the length of the capillary membranes is determined by the width of the mat. In a further preferred embodiment, the capillary membrane mat can be a woven mat in which the capillary membranes and the connecting fibers are woven together and in which the capillary membranes run in the extension direction or running direction of the capillary membrane mat, and the textile fibers run transverse thereto. Capillary membrane knitted and woven mats, as well as ways of producing them, are described, for example, in DE 38 39 567, DE 43 08 850 and EP 0 442 147. Such mat-like capillary membrane systems facilitate easy handling and easy introduction of the planar capillary membrane system into the interior spaces of the culture dishes. Moreover, such arrangements result in very uniform distribution of nutrient media, e.g., onto the cell culture in the respective culture dish or uniform disposal of degradation products from the cell culture.

For a uniform distribution of the cells in the interior space of the at least one cell culture dish, due to the mat-like construction, in particular for culture dishes having interior spaces with rectangular or square cross-sections, it is advantageous if the capillary membranes are at a uniform distance from one another. Preferably, the distance of the capillary membranes from one another in the mat is 1 to 10 times the external diameter of the capillary membranes, wherein the distance is measured from the longitudinal axes of the capillary membranes. A distance of 1.05 to 3 times the external diameter of the capillary membranes is particularly preferred. It is likewise advantageous if the connection elements are at a defined distance from one another, which distance preferably lies in the range of 1 to 20 mm, a distance in the range of 3 to 7 mm being preferred.

In the case of a spiral structure of the at least one capillary membrane system or in an arrangement of the capillary membranes in the capillary membrane system in mutually concentric circles with a view to uniform supply of the cells, it is likewise advantageous that the distances between the capillary membranes are constant, i.e. that the spiral whorls or the mutually concentric capillary membranes in the capillary membrane system are kept at the same distance from one another. This can likewise be accomplished using suitable connection elements, such as, e.g., connecting fibers, adhesive tapes or strand-like elements made of silicone material. As materials for the capillary membranes, all prior art organic polymers which are suitable for the formation of capillary membranes may in principle be considered, wherein said polymers must have good biocompatibility. It is moreover also required that the membrane polymer allows for sterilization of the device, for example by steam sterilization, sterilization by γ-irradiation or by ethylene oxide. In this connection, the organic polymers may be natural polymers or synthetically produced polymers. Natural polymers are in particular those based on cellulosic polymers, which likewise includes polymers that have been subjected to so-called polymer-analogous reactions.

Examples of polymers based on cellulose are those from regenerated cellulose, cellulose acetate or modified cellulose, such as, e.g., cellulose esters, cellulose ethers, cellulose modified with benzyl groups (benzyl cellulose) or cellulose modified with dimethylaminoethyl or mixtures of these cellulosic polymers. Furthermore, polymers based on chitin or chitosan may also be used.

As synthetically produced polymers, i.e. as synthetic polymers, those polymers can be used which consist of polyolefins, polyamides, polyacrylonitrile, polycarbonates, polyesters or sulfone polymers, and modifications, blends, mixtures or copolymers of these polymers obtained therefrom. Preferably, those polymers are used that are based on sulfone polymers, such as, in particular, polysulfone or polyether sulfone. As additives, further polymers may be admixed to these polymers, such as, e.g., polyethylene oxide, polyhydroxy ether, polyethylene glycol, polyvinyl alcohol or polycaprolactone. In addition, the capillary membranes may also be coated with an additive. Such capillary membranes preferably contain a hydrophilizing agent, e.g. polyvinylpyrrolidone, or hydrophilic modifications of these polymers, as well.

As has already been explained with regard to the embodiments of the device which have a cover for covering the opening of the interior space of the at least one culture dish, it is advantageous if the supply lines are connected to connector sleeves, via which they pass through the cover and are led out of the interior space. A simple connection can be established using such connector sleeves, e.g., to a reservoir for a nutrient solution or to a vacuum unit, by means of which, e.g., dissolved degradation products can be suctioned from the cell culture dishes. The connector sleeve is preferably a Luer lock connector, which makes a simple and clean connection possible.

In an advantageous embodiment, each supply line leading out of the interior spaces of the cell culture dishes is connected to a connector sleeve in the form of a Y-connector, which is connectable with its free ends, for example, to a fluid supply system and a removal system of the at least one capillary membrane system, i.e. by means of the Y-connector, an individual supply line is connectable to a liquid reservoir via a first sub-line and to a vacuum unit via a second sub-line. The connection can also be established using Luer lock connectors. It is possible to intermittently supply liquids or to suction off degradation products over predetermined time intervals by means of controllable shut-off valves.

The device according to the invention is characterized by a high degree of flexibility. For example, since the culture dishes are isolated from one another, in devices with a plurality of bowl-shaped culture dishes, different cell cultures can be grown in the individual bowl-shaped culture dishes without causing mutual interference of the cell cultures. The planar capillary membrane systems arranged in the interior spaces can moreover link their supply lines, independently of one another, with different supply or removal lines, so that the cell cultures colonized in the culture dishes can be supplied with various nutrient media, for example. It is also possible, for example, to provide different supplies to the different cell cultures with regard to nutrient solution concentrations or time intervals for supply. Moreover, it is also possible that differing capillary membrane system are arranged in the culture dishes of the device, which differ, for example, in their function or with regard to the characteristics of the capillary membranes used therein. Another advantage of the device according to the invention is that individual capillary membrane systems of the device can be exchanged, e.g., when the outside of the capillary membranes become overgrown.

When using the device according to the invention, an operational mode is also possible in which the cells for the cell culture are started, e.g., as a cell suspension, in the interior space of the at least one culture dish and then colonize the base. The supply system in the form of the planar capillary membrane system is subsequently placed thereupon.

The device according to the invention can also have at least one further capillary membrane system in one or in all of its culture dishes, which system assumes further tasks for growing cells. In addition to a capillary membrane system for supplying nutrient solution, at least one of the culture dishes of the device can have a further capillary membrane system with membranes for oxygenation, via which the cell culture can be supplied with oxygen in the interior space of the respective culture dish. At least one further capillary membrane system can also be included, via which degradation products can be disposed, so that supply and disposal can take place via different capillary membrane systems. It is also possible that, for example, two different capillary membrane systems are interwoven to form a mat, e.g., or knitted together using connecting threads to form a knitted mat. In these cases, the supply lines associated with the respective capillary membrane system are conveniently located at different sides of the capillary membrane mat thus formed.

The invention is further explained by means of the following figures, wherein the scope of the invention is not limited by the figures:

The following is shown:

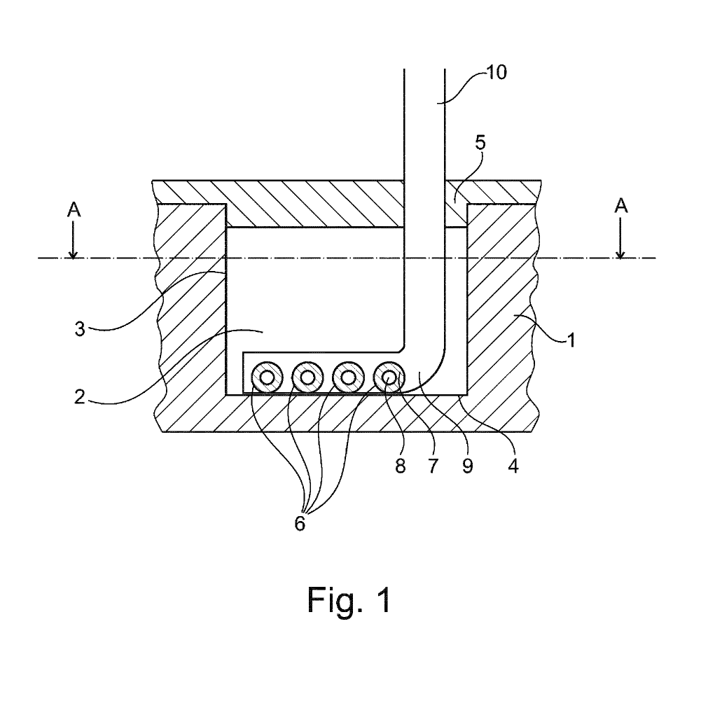

FIG. 1: a cross-section through a single bowl-shaped culture dish according to the invention.

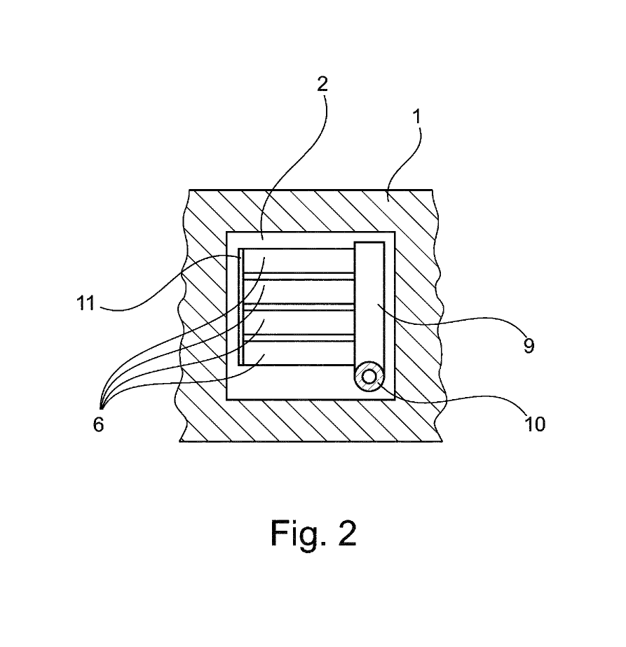

FIG. 2: viewed from above, a cross-section of the culture dish shown in FIG. 1 along the line A-A in FIG. 1.

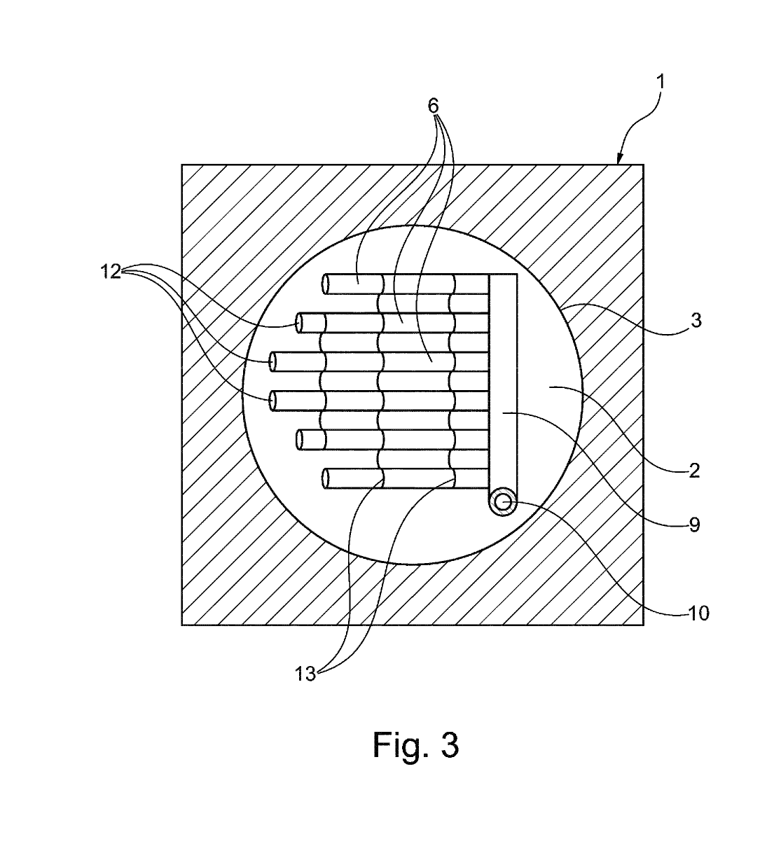

FIG. 3: viewed from above, a cross-section of a bowl-shaped culture dish having a round internal cross-section.

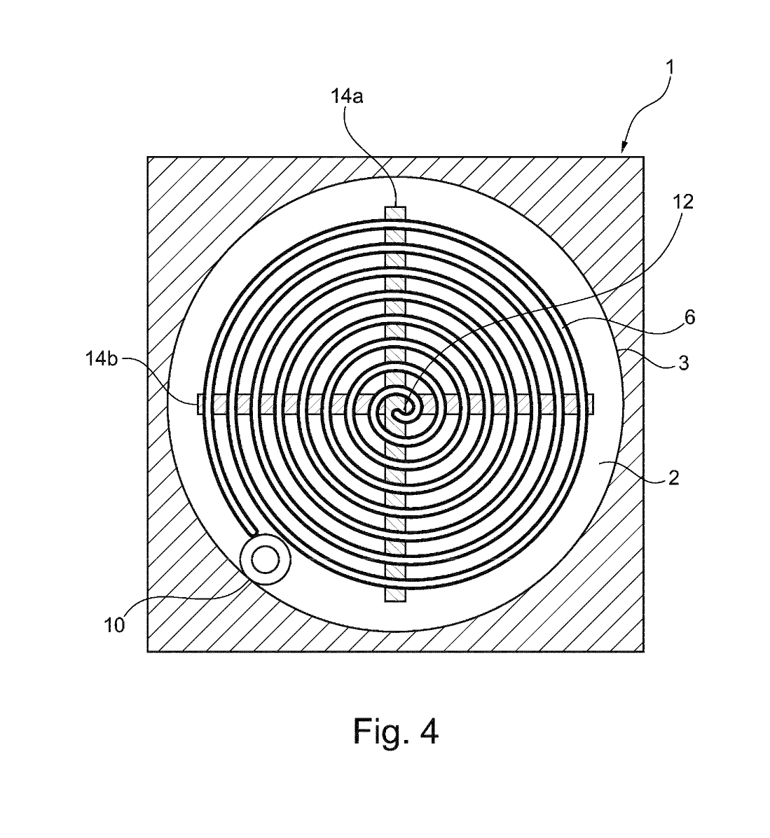

FIG. 4: viewed from above, a cross-section of a bowl-shaped culture dish having a round internal cross-section and a single capillary membrane, arranged in the region of the base of the culture dish and spirally designed to form a planar capillary membrane system.

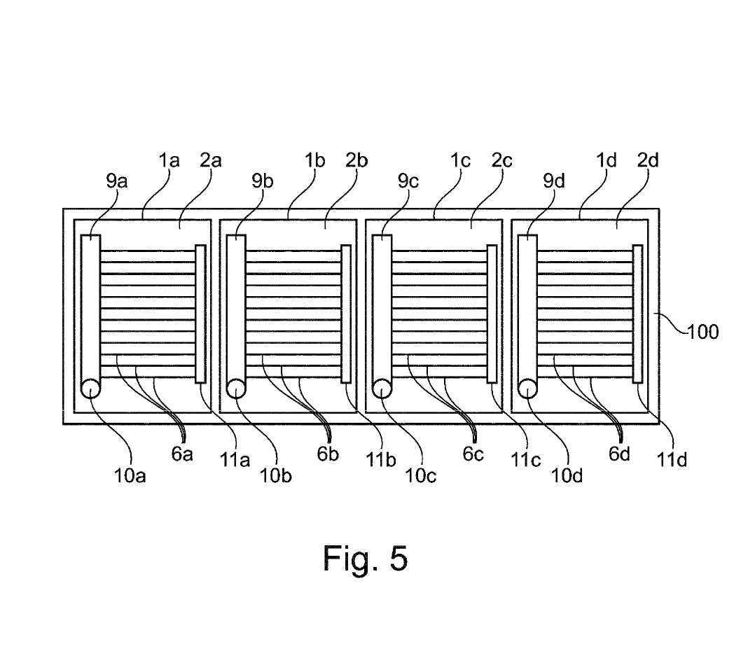

FIG. 5: viewed from above, a cross-section of a device with four bowl-shaped culture dishes arranged adjacent to one another, each having a rectangular cross-section, in each of which is arranged a planar capillary membrane system having a rectangular contour.

FIG. 1 shows a cross-section of a bowl-shaped culture dish 1 in a device 100, according to the invention, as it is suitable for growing and studying cells. The bowl-shaped culture dish 1 has an interior space 2 with a square cross-section bounded by a side wall 3 and a base 4. In this case, the opening of the interior space directed upwards is closed by a cover 5.

A planar capillary membrane system consisting of a plurality of mutually parallel capillary membranes 6 is arranged in the region of the base, wherein FIG. 1, for example, shows a cross-sectional view of four capillary membranes. Each of the capillary membranes has a semi-permeable wall 7 and a lumen 8 enclosed by the wall. With its rear end, as seen in the direction of view, the capillary membranes are embedded in a supply line 9 in such a way that a fluid connection exists between the supply line 9 and the lumens of the capillary membranes 6, so that fluids, media, gases and/or other substances can be conveyed through the supply line and the at least one capillary membrane system. With its vertical branch 10, the supply line leads out from the interior space 2 of the culture dish 1 through an opening in the cover 5 and can be connected to a supply unit, e.g. in the form of a pump or a reservoir, or to a disposal unit, e.g. in the form of a vacuum pump.

FIG. 2 is a cross-section of the bowl-shaped culture dish 1 shown if FIG. 1 along section line A-A. The planar capillary membrane system having a square cross-section in this example, arranged in the interior space 2, has a rectangular contour and is formed from mutually parallel capillary membranes 6. The capillary membranes 6 are embedded at one of their ends in a strand-like hot melt adhesive material 11, whereby the capillary membranes are closed at this end and are simultaneously held stably at a distance from one another.

On their other end, the capillary membranes are embedded in the supply line 9 which leads out of the interior space 2 of the bowl-shaped culture dish 1 via its upwards leading branch 10, as shown in FIG. 1.

In an enlarged scale, FIG. 3 shows a relevant cross-section of a bowl-shaped culture dish corresponding to FIG. 2, having a round interior cross-section. In this example, the planar capillary membrane system arranged in the interior space has a contour adapted to the contour of the interior space and is likewise formed from a plurality of mutually parallel capillary membranes. The length of the capillary membranes 6 is adapted to the contour of the internal cross-section, resulting in an arched contour at the edge formed by the free ends 12 of the capillary membranes 6 of the planar capillary membrane system. The capillary membranes 6 are closed at their free ends 12, e.g., by fusing or dipping in a hot melt adhesive. In this example, the planar capillary membrane system is designed to form a mat in which the capillary membranes 6 are connected to one another by thread-like connection elements 13 and are simultaneously held stably at a distance from one another.

With their second end, the capillary membranes 6 are embedded in the supply line 9 which leads out of the interior space 2 of the bowl-shaped culture dish 1 via its upwards directed branch 10 which is perpendicular to the plane of view.

FIG. 4 likewise shows a bowl-shaped culture dish 1 having a round internal cross-section. In this bowl-shaped culture dish 1, in the region of the base, a single capillary membrane 6 is spirally wound and thus formed into a two-dimensional capillary membrane system. In this embodiment, one of the ends of the spirally arranged capillary membrane 6 is open and connected to the supply line 10, which leads upwards out of the culture dish. The other, free, end 12 of the capillary membrane 6 is closed. The individual whorls of the spiral formed by the capillary membrane are held at a distance from one another and mutually stabilized by the connection elements 14a, b, e.g. in the form of adhesive tapes or strand-like elements made of silicone material.

FIG. 5 shows a schematic representation of a device 100 having four adjacent bowl-shaped culture dishes 1a-d, each having a rectangular cross-section. The bowl-shaped culture dishes 1a-d are isolated from one another and are not connected with one another. In the interior spaces 2a-d in each of said culture dishes 1a-d, a separate planar capillary membrane system having a rectangular contour is arranged, analogous to the culture dish shown in FIG. 2, which system is formed from a plurality of mutually parallel capillary membranes 6a-d. The capillary membranes 6a-d of the respective capillary membrane systems are embedded at their free end in a strand-like hot melt material 11a-d, whereby they are closed at this end and simultaneously held stably at a distance from one another.

With their other end, the capillary membranes 6a-d are embedded in respective supply lines 9a-d. Via their upwards directed branches 10a-d, the supply lines 9a-d are separately directed out of the culture dishes 1a-d through each opening at the top.

1. A device for growing and/or studying cells, comprising an arrangement of at least one bowl-shaped culture dish having a base and a side wall and an interior space which is bounded by base and side wall and which has an opening towards the top, characterized in that
at least one planar capillary membrane system consisting of at least one capillary membrane is arranged in the interior space of the at least one culture dish,
wherein the at least one capillary membrane has a semi-permeable wall, a lumen which is enclosed by a wall and at least one open end,
wherein the at least one capillary membrane with its at least one open end is fluidically connected to at least one supply line having a wall and a lumen, so that fluids, media, gases and/or other substances can be conveyed through the supply line and the at least one capillary membrane system,
wherein the at least one capillary membrane system is arranged in the region of the base of the at least one culture dish, and its two-dimensional extension is bounded by the side wall of the at least one culture dish, and
wherein the at least one supply line leads out of the interior space through the opening.

2. The device according to claim 1, characterized in that the at least one culture dish is provided with a cover which covers the opening of its interior space, and that the at least one supply line of the at least one culture dish passes through the cover.

3. The device according to claim 1, characterized in that the at least one planar capillary membrane system comprises a plurality of mutually parallel capillary membranes which are connected to one another to form a mat by means of a plurality of spaced and mutually parallel connection elements and are held at a distance from one another by the connection elements.

4. The device according to claim 3, characterized in that the distance of the capillary membranes from one another in the mat is 1.0 to 10 times the external diameter of the capillary membranes, wherein the distance is measured from the longitudinal axes of the capillary membranes.

5. The device according to claim 1, characterized in that the capillary membrane of the at least one capillary membrane system is connected to two supply lines, wherein the capillary membranes are each embedded in a common supply line with their opposite ends.

6. The device according to claim 1, characterized in that the at least one common supply line is a flexible silicone tube.

7. The device according to claim 1, characterized in that the supply lines leading out of the interior spaces of the cell culture dishes are each connected to a Y-connector which is connectable at its free ends to a fluid supply system and a disposal system.

8. The device according to claim 1, characterized in that the base of the at least one culture dish has an area in the range of 2 to 30 cm².

9. The device according to claim 1, characterized in that the area of the at least one planar capillary membrane system is 30 to 90% of the area of the base of the at least one culture dish.

10. The device according to claim 1, characterized in that it comprises at least two culture dishes which are isolated from one another and have no connection to one another and which are separately provided with at least one respective capillary membrane system.

11. The device according to claim 10, characterized in that it comprises at least six culture dishes.
